(19) United States
(12) Patent Application Publication
(10) Pub. No.: US 2010/0022018 A1
(43) Pub. Date: Jan. 28, 2010

(54) RAPID DETECTION OF SULFHYDRYL COMPOUNDS USING A PAPAIN BASED ASSAY

(75) Inventor: Dennis Flint, Newark, DE (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(73) Assignee: E.I. DU PONT DE NEMOURS AND COMPANY, Wilmington, DE (US)

(21) Appl. No.: 12/446,997

(22) PCT Filed: Nov. 28, 2007
(86) PCT No.: PCT/US07/24483

§ 371 Date: Apr. 24, 2009

Related U.S. Application Data
(60) Provisional application No. 60/861,460, filed on Nov. 29, 2006.

(51) Int. Cl. G01N 33/00; G01N 1/22

(52) U.S. Cl. 436/120; 422/85; 422/61

(57) ABSTRACT

The invention relates to a method for detecting compounds containing a free sulfhydryl group in a sample and a test device which is used to detect such compounds. The method and test device use a papain-based assay where the papain is initially complexed with a metal ion making it inactive. Sulfydryl compounds in a sample activate the papain. Enzymatic activity of papain on a papain substrate releases a dye producing a detectable signal. The test device may be used in detecting compounds containing a free sulfhydryl group in industrial, environmental, experimental and biological samples.

### FIELD OF THE INVENTION

This invention relates to the field of environmental sensors. Specifically, the invention relates to a rapid and sensitive assay for detection of sulfhydryl compounds capable of activating papain that is enzymatically inactive due to the binding of a metal ion to the sulfhydryl group of the papain's active site.

### BACKGROUND OF THE INVENTION

Hydrogen sulfide is a flammable, colorless gas that is toxic at extremely low concentrations, with potential of causing human fatalities. Detection of hydrogen sulfide is desired for industrial and environmental monitoring. For example, workers in the oil, gas, and petrochemical industries need protection from hydrogen sulfide exposure. Sulfur gases such as hydrogen sulfide (H₂S) and methylmercaptan (methanethiol) (CH₃SH), together with other volatile sulfur compounds (VSCs), including some thiols, organic sulfides and polysulfides, are often the cause of bad odors at sewage treatment and disposal works, and in other gaseous industrial effluents.

Volatile sulfur compounds such as hydrogen sulfide, methyl mercaptan, and dimethyl sulfide are also produced by oral bacteria causing halitosis. Unpleasant breath odor is not readily detected by the individual. It is generally detected by another individual, or by gas chromotographic analysis of volatile sulfur compounds present in the breath. A simple, rapid, sensitive and personal method of detecting halitosis would be useful as a personal care product.

Disclosed in Singh et. al. (Analytical Biochemistry 213: 49-56(1993)) is an assay for sulfhydryl groups which uses papain that has been inactivated by forming a disulfide linkage at the active-site sulfhydryl group. The assay is run for a minimum of 30 minutes and has low sensitivity.

There is a need for a rapid and highly sensitive method for detecting in a sample the presence or absence of at least one sulfhydryl compound. A method that an individual can use without the need for sophisticated laboratory equipment would be particularly useful.

### SUMMARY OF THE INVENTION

The present invention provides a method for detecting in a sample the presence or absence of at least one sulfhydryl compound, comprising:
(a) forming a detection mixture by contacting the sample with a papain-metal ion complex and a papain substrate having incorporated therein a non-peptidyl moiety, said moiety capable of being cleaved from the substrate by papain, said moiety having a light absorption or emission characteristic that changes depending upon whether the moiety is incorporated in the substrate or cleaved therefrom to form a released moiety in the detection mixture;
(b) detecting the presence or absence of the released moiety in the detection mixture; and
(c) correlating the presence or absence of the released moiety with the presence or absence of the sulfhydryl compound in the sample.

In a preferred embodiment, the metal ion is selected from the group consisting of Ag⁺, Au⁺, Cu⁺, Cu⁺⁺, Hg⁺⁺, Cd⁺⁺, Pb⁺⁺, Zn⁺⁺, Au⁺⁺⁺, Pt(IV), and combinations thereof.

In yet another embodiment the invention provides a test device for detecting in a sample the presence or absence of at least one sulfhydryl compound, said test device comprising a support having coated thereon or contained therein a papain-metal ion complex and a papain substrate having incorporated therein a non-peptidyl moiety, said non-peptidyl moiety capable of being cleaved from the substrate by papain, said non-peptidyl moiety having a light absorption or emission characteristic that changes depending upon whether the non-peptidyl moiety is incorporated in the substrate or cleaved therefrom.

### DETAILED DESCRIPTION

The present invention provides a method for rapidly detecting the presence of sulfhydryl compounds using a papain-based assay. Papain used in the method is enzymatically inactive due to the formation of a metal ion complex with the sulfhydryl group at the active site of the enzyme, resulting in a papain-metal ion complex. When the complex is contacted with a test sample containing a sulfhydryl compound, the inactive papain is rapidly and efficiently activated by removing the metal from the active site of papain. The enzymatic activity of papain then releases a non-peptidyl moiety from a papain substrate, to provide a released moiety that can be detected. In a preferred embodiment, the moiety is detected by optical means, such as a change in light absorption or emission.

Sulfhydryl compounds are present in a variety of samples including environmental, industrial, laboratory and biological samples. Detection of the sulfhydryl compounds in different samples is needed for reasons such as safety, experimentation, and personal care.

The following definitions and abbreviations are to be used for the interpretation of the claims and the specification.

The term “sulfhydryl compound” refers to a compound having at least one —SH group. Examples of sulfhydryl compounds include, but are not limited to, hydrogen sulfide, methyl mercaptan, cysteine, and dithiothreitol.

The term “papain-metal ion complex” refers to the enzyme papain having a metal ion complexed to the sulfhydryl group at the papain's active site, which causes the papain to be enzymatically inactive. The active site sulfhydryl group of papain is on the side chain of cysteine 25 in its amino acid sequence.

The term “contacting” as used herein, is intended to include any method whereby a sample suspected of containing sulfhydryl compounds is brought together with the papain-metal ion complex such that the papain can be enzymatically activated by sulfhydryl compounds that may be present in the sample.

The term “papain substrate” refers to a substrate that is cleaved by the enzymatic activity of papain.

The term “non-peptidyl moiety” refers to a compound that is non-proteinaceous and that has an amine or hydroxyl group that is capable of being covalently bound in an amide or ester linkage at the C-terminal end of a peptide of at least two amino acids. The resulting compound containing the peptide and the non-peptidyl moiety can fit into the active site of papain and be cleaved at the bond between the C-terminal end of the peptide and the non-peptidyl moiety, i.e., it is a papain substrate.

The term “dye” refers to a compound that produces a detectable signal using an optical assay. Typically a dye produces a signal through properties such as fluorescence, or light absorption.

### Papain-Based Assay for Sulfhydryl Compounds

Papain contains a sulfhydryl group in the active site that is essential for its enzymatic activity. The active site sulfhydryl group is on the side chain of cysteine 25 in the papain amino acid sequence. Blockage of the active site sulfhydryl group such as by forming a disulfide linkage or complexing it with a tightly bound metal blocks the enzymatic activity of papain. Freeing the sulfhydryl group from this blocked state allows papain to regain its enzymatically active state. The activated papain is able to cleave a papain substrate, whereas the papain substrate is uncleaved in the presence of blocked papain. Sulfhydryl compounds are known to be able to unblock the active site sulfhydryl group of papain that is in a disulfide linkage (Singh et. al. Analytical Biochemistry 213: 49-56 (1993)) or bound to Hg⁺⁺ (Kimmel and Smith, Journal of Biological Chemistry, 207: 515-31 (1954); Smith, Kimmel, and Brown, Journal of Biological Chemistry, 207: 533-49 (1954))

Surprisingly, applicant has found that sulfhydryl compounds are detected with much higher sensitivity in an assay that uses papain having the active site sulfhydryl group complexed with a metal ion as compared to papain having the active site sulfhydryl group in a disulfide linkage. For example, applicant found that in a one minute assay 100 parts per billion (ppb) of gaseous hydrogen sulfide was detected with over 10-fold greater sensitivity using papain inactivated by Ag⁺ as compared to papain inactivated because its active site sulfhydryl group was in a disulfide linkage. In addition, applicant found that in a one minute assay 100 ppb of gaseous hydrogen sulfide was detected with over 50-fold greater sensitivity using papain complexed with Hg⁺⁺ as compared to papain inactivated because its active site sulfhydryl group was in a disulfide linkage. Thus the instant invention makes use of this highly sensitive activation of a papain-metal ion complex for detecting sulfhydryl compounds.

In the present method, papain in a papain-metal ion complex is activated by contact with a sulfhydryl compound, and the activated papain cleaves a non-peptidyl moiety from a papain substrate. Release of the non-peptidyl moiety is detected by a change in properties of that moiety upon cleavage, as described below. Since one reactivated papain enzyme can catalyze the cleavage of many papain substrate molecules, the response of this assay is amplified.

### Papain-Metal Ion Complex

In the instant invention, papain may be complexed with any metal ion which blocks the active site sulfhydryl group thereby disrupting its enzymatic activity, and which is readily removed from the papain-metal ion complex in the presence of a sulfhydryl compound. Particularly useful are the metal ions Ag⁺, Au⁺, Cu⁺, Cu⁺⁺, Hg⁺⁺, Cd⁺⁺, Pb⁺⁺, Zn⁺⁺, Au⁺⁺⁺, and Pt(IV). Different metal ions provide different levels of inhibition of papain enzymatic activity. Particularly desirable are metal ions causing inhibition such that minimal papain activity remains when papain and the metal ion form a complex, and that are readily removed from the papain-metal ion complex in the presence of a sulfhydryl compound. In this regard, more particularly useful are Hg⁺⁺ and Ag⁺.

The metal ion is added to a solution of papain in the form of a salt. For example, for the metal ions listed above, AgNO₃, AuCl, Cu(C₃H₅N)Cl, CuCl₂, HgCl₂, Cd(NO₃)₂, Pb(C₂H₃O₂)₂, ZnCl₂, AuCl₃, Pt(IV)Cl₄ may be added, respectively. In addition, a mixture of metal ions may be used to inhibit papain. An amount and type of metal ion is added which inactivates papain providing the reduced background enzyme activity required for the specific assay being used. Generally it is desired to have a very low background papain activity level to provide a sensitive assay. Inhibition of papain by over 99% is generally achieved by adding about 1 to about 1.5 molar equivalent of metal salt to it.

Additional considerations for choosing a metal ion for papain inactivation are the properties of the metal ion itself and the particular use of the assay. For example, Hg⁺⁺ is extremely efficient in papain inactivation and reactivation, as noted above. However, the toxicity of Hg⁺⁺ prohibits its use in certain assay situations, particularly where the user may come in contact with the papain-metal ion complex.

### Papain Substrate

Papain is a proteolytic enzyme with wide specificity, and has the ability to degrade most proteins. Papain catalyzes the breakdown of proteins by cleaving the amide bonds between amino acids. Artificial substrates that may be used for detecting papain activity generally include a peptide of two or more amino acids covalently bound at the C-terminal end to a non-peptidyl moiety. An additional component, which may provide a protecting group, may optionally be present at the N-terminus of the amino acid chain. Such an artificial substrate may be depicted as in Formula I:

Y-Aₙ-A₂-A₁-Z

wherein A₁ , A₂, and Aₙ are amino acids in a peptide chain, n typically being less than 5, Z is a non-peptidyl moiety, and Y is an optional linked compound or group.

Amino acids A₁ and A₂ in Formula I are any combination of amino acids that are recognized by papain such that the bond between A₁ and Z is readily cleaved by the enzymatic activity of papain. Additional amino acids at the N-terminus of A₂ may contribute to papain recognition. Different combinations of amino acids in the A₁ and A₂ positions are recognized by papain with different efficiencies. At position A₁, amino acids with a positive charge such as lysine or arginine are particularly suitable. Most suitable is arginine in position A₁. At position A₂, hydrophobic or aromatic amino acids such as leucine or phenylalaine are particularly suitable. Most suitable is phenylalanine. An optional component at the N-terminus of the peptide, Y, may be any linked group or compound that may be included to provide substrate characteristics such lack of charge on the N-terminus, improved stability, or ease of synthesis or purification.

The non-peptidiyl moiety of the papain substrate has an amine or hydroxyl group and forms an amide or ester with the carboxyl group of the A₁ amino acid. Amines are preferred since the ester bond formed from a hydroxyl on the non-peptidiyl moiety is less stable than the bond formed with the amine. Amide hydrolysis by papain enzymatic activity cleaves the A₁-Z bond, releasing the non-peptidiyl moiety. The non-peptidyl moiety of the papain substrate is generally chosen so that upon cleavage, it undergoes a change in properties that can be easily detected. Particularly useful in the present method are papain substrates having a non-peptidyl moiety that when released from the papain substrate, has a change in light absorption or emission characteristics. The change in characteristics may be detected using experimental equipment or by the senses, which indicate papain activity. For example, a non-peptidyl moiety when bond in a papain substrate may have no fluorescence activity, but following release it exhibits fluorescence. Fluorescent signals may be detected by a fluorometer, as is well known to one skilled in the art. Examples of papain substrates which produce a fluorescent signal when cleaved include, but are not limited to, those having amino methyl coumarin (AMC) such as CBZ-F-R-AMC (Bachem Bioscience, Inc; King of Prussia, Pa.), or ethylene diamaine dinitrophenol (Dnp) such as Abz-Gln-Val-Val-Ala-Gly-Ala-ehtylenediamine-Dpn (Bachem Bioscience, Inc).

A particularly useful papain substrate for the present method is one having a non-peptidyl moiety that has changes in absorption upon release by papain. The non-peptidyl moiety may have a change in absorption at one or more wavelengths following release. For example, an increase in absorption of a specific wavelength may be measured over time as the non-peptidyl moiety is released by papain activity. The light absorption signals may be detected using a spectrophotometer or visually. Particularly useful is an increase in absorption of a wavelength that causes a color change that can be detected visually. Examples of papain substrates which produce a visible signal when cleaved include, but are not limited to, those having p-nitroanaline (pNA) or dinitrophenyl (Dnp) groups such as Ac-Phe-Gly-pNA ((Bachem Bioscience, Inc, O-aminobenzoyl (Abz)-QVVAGA-ethylenediamine-2-4-dinitrophenyl (Bachem Bioscience, Inc), and Bz-Phe-Val-Arg-pNA (Bachem Bioscience, Inc). Particularly useful in the instant invention is the substrate Z-F-R-pNA (N-[(phenylmethoxy)carbonyl]-L-phenylalanyl-N-(4-nitrophenyl)-L-argininamide; Bachem Biosciences Inc., King of Prussia, Pa.). Z-F-R-pNA is colorless, and papain activity releases the non-peptidyl moiety nitroanilide, which is yellow in color, has an extinction coefficient of 8800 M⁻¹cm⁻¹ at 410 nm, and is visible to the naked eye. Papain substrates are commercially available, such as those exemplified above. In addition, new substrates may be developed which have the property of producing a detectable change in characteristics of the non-peptidyl moiety upon release by papain, and these may be used in the instant invention. Substrates may vary in their susceptibility to cleavage by papain. More readily cleaved substrates provide greater amplification for the detection of sulfhydryl compounds. For example, one molecule of papain can catalyze the hydrolysis of 150-600 molecules of a good papain substrate per minute, providing an amplification factor in the hundreds fold range.

### Samples

Samples that may be assayed to detect the presence of a sulfhydryl compound include any substance suspected of containing one or more sulfhydryl compounds. Samples may be obtained, for example, from a chemical synthesis, an industrial source, an environmental source, or an animal source. Samples may be in solution or may be gaseous. Oil wells, oil drilling locations, and natural gas facilities are environments of interest for sample collection. Of particular interest are human breath samples. In one embodiment, the method of this invention is used to diagnose halitosis in an individual, the sample being a breath sample from the individual and the presence of the sulfhydryl compounds in the sample being indicative of halitosis.

### Assay Conditions

Samples in solution and gaseous samples containing sulfhydryl compounds may be assayed using any conditions under which the papain enzyme, once activated, is able to cleave a papain substrate. Such conditions are well known to one skilled in the art. For example, papain is active in a pH range of about 5 to about 9. Gaseous samples of sulfhydryl compounds are more effectively assayed at more basic pH within this range, since these compounds reach a higher concentration in aqueous solutions at higher pHs. Thus assay of gaseous samples is more effective at a pH of about 8 than a pH of 6. The papain-metal complex may be in solution in a reaction buffer containing a buffering substance for maintaining the desired pH, typically Tris or MOPS. Alternatively, the papain-metal ion complex and reaction buffer may be dried.

Sensitivity of the sulfhydryl compound assay is related to the amount of active papain present in the assay reaction. Therefore, for highest sensitivity it is desirable to include papain at a concentration that is near its solubility limit, which is between about 10⁻⁵ and about 10⁻⁴ molar. In many applications it is desirable that the detection be rapid, such as in breath analysis or in assessing the safety of an environment. Maximal papain concentration in the assay provides maximal speed of the assay. Lower concentrations of papain may be used with reduced sensitivity and slower detection times.

To initiate the assay, the test sample suspected of containing sulfhydryl compounds is brought in contact with the papain-metal ion complex. Contact may be made by mixing a test sample in solution with a solution containing the papain-metal ion complex, by the flow of a gaseous test sample over papain-metal ion complex in solution or dry, or any combination thereof that provides access of a sulfhydryl compound to the papain-metal ion complex. When dry papain-metal ion complex is provided for contact with a non-liquid sample, a humectant may be included in the dry sample. The humectant absorbs moisture, such as from a breath sample, thereby providing a liquid environment for the papain reaction. Examples of humectants include glycerine, propylene glycol, glyceryl triacetate, polyols such as sorbitol, xylitol, and maltitol, and polymeric polyols such as polydextrose.

Activated papain resulting from contact with a sulfhydryl compound then reacts with a papain substrate which may be added in a second step, or concurrently with the papain-metal ion complex. The papain substrate is one that produces an assayable signal upon cleavage as described above. The signal is then detected using an instrument for detecting signals such as fluorescence, or light absorption as is well known to one skilled in the art. Preferably the signal is detect by visual inspection so that use of instrumentation is not required.

### Sulfhydryl Compound Test Device

A s test device for detecting sulfhydryl compounds such as hydrogen sulfide (H₂S), methylmercaptan (methanethiol) (CH₃SH), organic sulfides and polysulfides includes a papain-metal ion complex, a papain substrate having a peptide bonded to a non-peptidyl moiety whose light absorption or emission characteristics change if the non-peptidyl moiety is released from the papain substrate, and a reaction buffer. These components are as described above. In the test device, the components may be in separate containers, to be mixed by the user, or mixed in one container. The components may be in a liquid state, or in a dry state. In a particularly useful format, the components are dried on a surface. A particularly useful surface is a disposable strip. On the surface of the strip are papain-metal ion complex, papain substrate and reaction buffer, providing a test strip for assay of samples suspected of containing sulfhydryl compounds. Such a test strip is placed in a moisture-proof covering. Prior to sample exposure, the covering is removed and the strip is wetted. Alternatively, a humectant in the components on the strip absorbs moisture from the environment so no direct wetting is needed.

The test strip, or other format of the test device, is used for the detection of sulfhydryl compounds in industrial, environmental, experimental, and biological samples. Samples from oil wells, oil drilling and natural gas locations, industrial effluents, laboratories or animals may be collected and contacted with a test device of the invention. For example, for detection of sulfhydryl compounds in a breath sample, a test device that is a support having coated thereon or contained therein a papain-metal ion complex and a papain substrate having incorporated therein a non-peptidyl moiety that is a dye. The test device is optionally wetted with water or saliva. Preferably, a humectant on the test device absorbs moisture from a breath sample that is passed over the support for a period of about 20 to 60 seconds. Any sulfhydryl compounds present in the breath sample diffuse into the liquid on the test device, remove the metal ions from the papain-metal ion complex, and thereby activate the papain enzymatic activity. This activity cleaves the dye from the substrate, whereby a light absorption or emission characteristic of the dye changes resulting in a color change on the test device that is detectable by the human eye.

The test device, or test devices, may be provided in a personal hygiene kit in combination with breath remediating supplies such as chewing gum, breath mints, or breath strips. Particularly suitable is a kit that includes at least one piece of chewing gum and at least one test device for assaying sulfhydryl compounds in human breath.

### EXAMPLES

The present invention is further defined in the following examples. It should be understood that these examples are given by way of illustration only. From the above discussion and these examples, one skilled in the art can ascertain the essential characteristics of this invention, and without departing from the spirit and scope thereof, can make various changes and modifications of the invention to adapt it to various usage and conditions.

### Materials

The papain used in these experiments was purchased from the Sigma Chemical Company (St. Louis, Mo.). Papain was assayed using the substrate N-[(phenylmethoxy)carbonyl]-L-phenylalanyl-N-(4-nitrophenyl)-L-argininamide (CA # 117761-01-0). This substrate was purchased from Bachem (Catalogue # L-1242; Bachem Biosciences Inc., King of Prussia, Pa.) and is abbreviated Z-F-R-pNA. A 10 mM stock solution of the substrate was made in DMSO.

The meaning of abbreviations is as follows: “mM” means millimolar, “pmol” means picomoles, “nmol” means nanomoles, “μl” means microlitre, ‘ppb’ means parts per billion, “ppm” means part per million, “Δ” means change in.

### Example 1

### Preparation of Inactive Papain Reagent

### Preparation of Disulfide Linked Inactivated Papain

A 7.7×10⁻⁴ M papain suspension purchased from Sigma was only partially active. The suspension was diluted 10 fold into 50 mM acetate buffer pH 4.1 to dissolve the papain. Next the papain was completely activated by adding 1.2 molar excess of dithiothreitol (DTT) and incubating for 30 minutes at room temperature. The DTT was removed by gel filtration using Sephadex G-25. The papain was then inactivated by adding of one of the following reagents 1) methyl methanethiolsulfonate, 2) 2,2′-dithiobis-pyridine, or 3) 4,4′-dithiobis-pyridine. Typically papain at 7×10⁻⁵ M was incubated with these reagents at 3.5×10⁻⁴ M for 1 hour at 0° C. The reagents used for inactivation were then removed by gel filtration using Sephadex G-25. Papain inactivated by one of these reagents was then used in the detection of low amounts of sulfur containing compounds in the examples below.

### Preparation of Metal Inactivated Papain

Papain was activated as described above, and the DTT was removed by gel filtration using G-25. Papain was then inactivated by adding a slight molar excess of a mono, di, tri, or tetra valent metal. Aliquots of 1 mM solutions of AgNO₃, AuCl, Cu(C₃N)₄NO₃, CuCl₂, HgCl₂, CdCl₂, PbCl₂, ZnCl₂, PtCl₂, AuCl₃, or Pt(IV)Cl₄ were added separately to 7.7×10⁻⁵ molar papain solutions such that a 1.5 molar excess of metal was added. The inhibition by the metals was essentially instantaneous. Papain inactivated by one of these metals was then used in the detection of low amounts of sulfur containing compounds in the examples below.

### Example 2

### Comparative: Poor Detection of Low Concentrations of Sulfide Compounds in Solution Using Papain Inactivated by Disulfide Linkage

The object of this experiment was to determine the sensitivity of disulfide linkage inactivated papain for detection of low of amounts of sulfur containing compounds. The molar amount of papain added to the detection assay was greater than the amount of sulfur containing compounds that were being detected so that the amount of papain would not be rate limiting.

To check the maximum sensitivity of an assay based on papain inactivated with reagents that formed a disulfide linkage with its active site sulfhydryl group, DTT was used as an activator because it is one of the most effective activators of papain known. 1 mM DTT solutions were made fresh in oxygen free water, and diluted to lower concentrations in oxygen free water in a glove box. Sealed solutions of DTT were removed from the glove box and opened just before use. The final volume of the solution based assay was 200 μl and contained a final concentration of approximately 50 mM Tris buffer, pH 8.0.

The assay was started by mixing 30 μl of a 7.7×10⁻⁵ M papain solution (23 nmoles of papain), 1-10 μl of a 1×10⁻⁵ M DTT solution (i.e., 10 to 100 picomoles of DTT), and sufficient buffer to add up to 100 μl. This mixture was incubated at 25° C. for 1 minute after which 100 μl of 1×10⁻⁴ M solution of Z-F-R-pNA in 50 mM Tris buffer, pH 8.0 was added and the change in absorbance at 410 nm immediately after mixing was measured with a Cary 4 UV-visable spectrophotometer. Since active papain releases p-nitroanaline on hydrolysis of Z-F-R-pNA, which absorbs at 410 nm, the activity of the papain was measured by following the changes in absorbance at 410 nm.

The rates of change in absorbance at 410 nm for different amounts of DTT are given in Table 1. These results indicated that activation using 10 picomoles of DTT of disulfide linkage inactivated papain gave a detectable change in absorbance.

TABLE 1
Reactivation of papain inactivated by
methyl methanethiolsulfonate with DTT

| Picomoles of DTT added | Δ A410/min |
|---|---|
| 10 | 0.52 |
| 20 | 0.96 |
| 50 | 1.9 |

Under the conditions of substrate, pH, and temperature used in our assays, each molecule of active papain would be expected to hydrolyze around 600 molecules of substrate per minute. Based on an extinction coefficient of 8800 M⁻¹ cm⁻¹ for p-nitroanaline, and an absorbance change of 1.9/min (obtained when 50 pmoles of DTT was added), the rate of appearance of p-nitroanaline was 2.6×10⁻⁴ M/min. At a turnover of 600/min it would take 3.6×10⁻⁷ M papain to generate product at this rate. 200 μl of a 3.6×10⁻⁷ M solution of active papain would contain 72 pmoles of papain, which is more than the molar amount of DTT added to the assay that produced an absorbance change of 1.9/min. This indicates that either the addition of DTT led to the activation of more than 1 molecule of papain per molecule of DTT (which is theoretically possible since it contains 2 free sulfhydryl groups) or the turnover number of papain under the conditions of our assay is somewhat more than 600/min.

The sensitivity of disulfide linkage inactivated papain for detection of low amounts of cysteine and sodium sulfide was also measured under very similar conditions to those used for DTT, except in some experiments higher concentrations of cysteine and sodium sulfide were used than was the case for the experiments with DTT. Otherwise the cysteine solutions were similarly prepared in and diluted with oxygen free water and kept sealed until seconds before using. Sodium sulfide was similarly prepared and diluted except oxygen free 0.1 N sodium hydroxide was used instead of water. The rate of change in absorbance at 410 nm after the incubation of various amounts of these compounds with inactivated papain for 1 min are given in Table 2.

TABLE 2
Reactivation of papain inactivated by methyl methanethiolsulfonate
with Cysteine and Sodium Sulfide

| | Δ A410/min |
|---|---|
| Picomoles of Cysteine added | |
| 20 | 0.45 |
| 50 | 1.1 |
| Picomoles of Sodium Sulfide added | |
| 50 | 0.12 |
| 100 | 0.27 |
| 200 | 0.77 |

Comparing these results with those obtained with DTT, shows that the activation of disulfide linkage inactivated papain by cysteine was not as efficient as with DTT. Apparently somewhat less that 1 molecule of papain was activated for each molecule of cysteine. The efficiency of activation by sodium sulfide was even lower, with on the order of one molecule of papain being activated for every 5 molecules of sodium sulfide.

Detection of cysteine and sodium sulfide was also tested using papain inactivated with 2,2′-dithiobis-pyridine, 4,4′-dithiobis-pyridine and methyl methanethiolsulfonate. The background level of activity exhibited by the papain inactivated by 2,2′-dithiobis-pyridine and 4,4′-dithiobis-pyridine was higher than papain inactivated with methyl methanethiolsulfonate, indicating that papain was not inactivated with 2,2′-dithiobis-pyridine and 4,4′-dithiobis-pyridine as completely as it was with methyl methanethiolsulfonate. However, the reactivation of papain inactivated by 2,2′-dithiobis-pyridine, and 4,4′-dithiobis-pyridine by cysteine and sodium sulfide was somewhat more efficient than it was with papain inhibited by methyl methanethiolsulfonate. The relatively high background of functional papain remaining in samples inactivated with 2,2′-dithiobis-pyridine, and 4,4′-dithiobis-pyridine made papain inactivated with these reagents less suitable for assays of sulfhydryl compounds than papain inactivated with methyl methanethiolsulfonate. Since the concentration of inactivated papain used in these assays was high to optimize the rate of the second order reaction between the activating sulfhydryl group and the inactivated papain, even small percentages of active enzyme gave backgrounds of activity that were significant compared to what was reactivated by the sulfhydryl reagents.

### Example 3

### Comparative: Detection Level of Gaseous Hydrogen Sulfide with Disulfide Linkage Inactivated Papain

In these experiments 200 μl of 50 mM Tris buffer, pH 8.0. and papain inactivated with methyl methanethiolsulfonate, as described in Example 1, were exposed to different concentrations of gaseous hydrogen sulfide mixed with nitrogen. A standard mixture of H₂S (10 ppm) in nitrogen was mixed with pure nitrogen gas using a gas mixing system that included calibrated flowmeters (Porter Instrument Company; Hatfield, Pa.) to create mixtures of H₂S at different concentrations. The flow rate of the H₂S containing gas mixtures was 1 liter/min. 3 mm ID Teflon tubing was attached to the exit port of the mixing device through which the gas mix was transferred to the assay tube. The open end of the Teflon transfer tubing was placed about 5 mm away from the surface of the solution containing the inactivated papain in 100 μl and at approximately a 45° angle to it. After one minute, 100 μl of substrate was added and the change in absorbance at 410 nm was measured in a spectrophotometer. Results for papain inactivated with methyl methanethiolsulfonate are shown in the table below.

TABLE 3
Detection of H₂S using disulfide linkage inactivated papain

| | Δ A410/min after a 1 minute |
|---|---|
| Concentration of H₂S in gas mix | exposure to gas mix |
| 100 ppb | 0.07 |
| 250 ppb | 0.21 |
| 500 ppb | 0.39 |
| 1 ppm | 0.74 |
| 2 ppm | 1.2 |

The results in this example show that papain inactivated because its active site thiol was in a disulfide linkage could at short time just barely detect H₂S at the levels found in bad breath (100-200 ppb).

### Example 4

### Highly Sensitive Detection of Gaseous Hydrogen Sulfide using Papain Inactivated with Hg⁺⁺

The detection of gaseous H₂S using papain that had been inactivated by Hg⁺⁺ ions was investigated as follows. Papain that was initially activated with DTT as described in Example 1 was passed through a G-25 column to remove the DTT. Enough HgCl₂ was added to equal 1.1× the molar amount of papain. This inhibited >99.9% of the activity of the activated papain. This papain was used to assay H₂S as follows. 100 μl of a solution containing 120 pmole of Hg⁺⁺ inactivated papain in 50 mM MOPS buffer, pH 8.0 was exposed to a stream of gas containing various concentrations of H₂S as described in Example 3. After 1 min exposure to the gas stream, 100 μl of a 5×10⁻⁴ M solution of the substrate, Z-F-R-pNA, was added and the change in absorbance at 410 nm immediately after mixing was measured with a Cary 4 UV-visable spectrophotometer. Results are shown in Table 4 below.

TABLE 4
Detection of gaseous H₂S using papain inactivated with Hg⁺⁺

| | Δ A410/min after a 1 minute |
|---|---|
| Concentration of H₂S in gas mix | exposure to gas mix |
| 0 ppb | 0 |
| 10 ppb | 1.9 |
| 100 ppb | >4 |
| 250 ppb | >4 |
| 500 ppb | >4 |

These results indicated that the strong inhibition of papain by Hg⁺⁺ was readily reversed in the presence of H₂S, which allows papain inhibited by Hg⁺⁺ to be used as a very sensitive and rapid H₂S sensor.

It should be noted that the amount of Hg⁺⁺ inhibited papain added to these assays was less than 1/100 that used in the assays of sulfhydryl compounds using papain inhibited because its active site sulfhydryl group was in a disulfide linkage. Successful assays for low levels of H₂S (and by inference other sulfhydryl compounds) require much less metal inhibited papain than papain inhibited because its active site sulfhydryl group is in a disulfide linkage. The lower the amounts of inhibited papain used, the smaller is the problem of background activity due to residual uninhibited papain.

### Example 5

### Very Sensitive Detection of Gaseous Hydrogen Sulfide Using Papain Inactivated with Ag⁺

The detection of gaseous H₂S using papain that had been inactivated by Ag⁺ ions was investigated as follows. Papain, initially activated with DTT as described is Example 1, was passed through a G-25 column to remove the DTT.

Enough AgNO₃ was added to equal 1.1× the molar amount of papain. This inhibited >99.5% of the activity of the activated papain. The Ag⁺ inactivated papain was used to assay H₂S in the same way the Hg⁺⁺ inhibited papain was used in Example 4. Results are shown in Table 5 below.

TABLE 5
Detection of gaseous H₂S using papain inactivated by Ag⁺

| | Δ A410/min after a 1 minute |
|---|---|
| Concentration of H₂S in gas mix | exposure to gas mix |
| 0 ppb | 0.01 |
| 100 ppb | 1.2 |
| 250 ppb | 2.3 |
| 500 ppb | >4 |
| 1 ppm | >4 |

The inhibition of papain by Ag⁺ was readily reversed in the presence of H₂S, which allows papain inhibited by Ag⁺ to be used for a sensitive and rapid H₂S sensor. For this reason, papain inhibited by Ag⁺ provided the basis of a very sensitive assay for H₂S. For example, Ag⁺ inactivated papain could be for the rapid detection of the levels of H₂S found in bad breath.

### Example 6

### The Detection of Gaseous Hydrogen Sulfide using a Dry-Support Type Test Device

120 pmole of Ag⁺ inactivated papain, prepared as described in Example 5, is added to 100 μl of 50 mM MOPS buffer that is 20% in glycerol and applied to a 0.75 cm² piece of filter paper attached to one end of a 0.75 cm×3 cm disposable strip. This test strip is air dried and placed in a moisture-proof covering. Multiple test strips are prepared. Prior to sample exposure, the covering is removed. On one test strip, the filter paper with dried sample is wetted by directly adding a drop of water. On another test strip, the filter paper becomes moist through the glycerol humectant's absorption of moisture from the air. A stream of gas containing 200 ppm of H₂S, prepared and applied as described in Example 3, is passed over the filter papers for 1 minute. The location on each filter paper where the reaction mix is applied turns yellow.

What is claimed is:

1. A method for detecting in a sample the presence or absence of at least one sulfhydryl compound, comprising:
(a) forming a detection mixture by contacting the sample with a papain-metal ion complex and a papain substrate having incorporated therein a non-peptidyl moiety, said moiety capable of being cleaved from the substrate by papain, said moiety having a light absorption or emission characteristic that changes depending upon whether the moiety is incorporated in the substrate or cleaved therefrom to form a released moiety in the detection mixture;
(b) detecting the presence or absence of the released moiety in the detection mixture; and
(c) correlating the presence or absence of the released moiety with the presence or absence of the sufhydryl compound in the sample.

2. The method of claim 1 wherein the metal ion is selected from the group consisting of Ag⁺, Au⁺, Cu⁺, Cu⁺⁺, Hg⁺⁺, Cd⁺⁺, Pb⁺⁺+, Zn⁺⁺, Au⁺⁺⁺, Pt(IV), and combinations thereof.

3. The method of claim 2 wherein the metal ion is Hg⁺⁺ or Ag⁺.

4. The method of claim 1 wherein step (b) is carried out by detecting the presence or absence of the released moiety by optical means.

5. The method of claim 4 wherein the optical means comprises determining whether or not there is a change in the absorption by the detection mixture of light of at least one preselected wavelength.

6. The method of claim 5 wherein the change in absorption is visually observable as a color change of the detection mixture.

7. The method of claim 4 wherein the papain substrate is nonfluorescent, and the released moiety is fluorescent, and step (b) is carried out by determining whether the detection mixture exhibits fluorescence.

8. The method of claim 4 wherein said papain-metal ion complex and said papain substrate are disposed in or on a solid support of a test device.

9. The method of claim 8 wherein the sample is human breath.

10. The method of claim 9 wherein said contacting step (a) comprises exhaling said breath onto said device.

11. A test device for detecting in a sample the presence or absence of at least one sulfhydryl compound, said test device comprising a support having coated thereon or contained therein a buffer, a papain-metal ion complex and a papain substrate having incorporated therein a non-peptidyl moiety, said non-peptidyl moiety capable of being cleaved from the substrate by papain, said non-peptidyl moiety having a light absorption or emission characteristic that changes depending upon whether the non-peptidyl moiety is incorporated in the substrate or cleaved therefrom.

12. The test device of claim 11 wherein the non-peptidyl moiety is a dye.

13. The test device of claim 12 wherein additionally a humectant is dried on the support.

14. A kit for freshening the breath of a human being, comprising the test device of claim 12 or 13 and at least one piece of chewing gum that is capable of imparting a pleasant odor to the breath of the human being.
